(19) United States
(12) Patent Application Publication
(10) Pub. No.: US 2023/0000440 A1
(43) Pub. Date: Jan. 5, 2023

(54) Heartrate Detection Device, Heartrate Detection Method, and Heartrate Detection Program

(71) Applicant: Nippon Telegraph and Telephone Corporation, Tokyo (JP)

(72) Inventors: Yuki Hashimoto, Tokyo (JP); Kei Kuwabara, Tokyo (JP); Nobuaki Matsuura, Tokyo (JP)

(21) Appl. No.: 17/781,184

(22) PCT Filed: Dec. 4, 2019
(86) PCT No.: PCT/JP2019/047438

§ 371 Date: May 31, 2022

(51) Int. Cl. A61B 5/00; A61B 5/0245; A61B 5/352

(52) U.S. Cl. CPC A61B 5/7207; A61B 5/0245; A61B 5/352

(57) ABSTRACT

A heartbeat detection device includes: a sensor data acquisition unit that acquires sensor data indicating an electrocardiographic waveform of a living body and outputs a sampling data sequence D(i) based on the sensor data; a first calculation unit that calculates a temporal difference value DY(i) of sampling data D(i) from the output sampling data sequence D(i) for each sampling time; a determination unit that determines a heartbeat time on the basis of a time at which a change in the calculated temporal difference value DY(i) exceeds a set threshold; a correction unit that corrects the heartbeat time by using a reference time; and a heart rate calculation unit that calculates a heart rate of the living body from the corrected heartbeat time.

### CROSS-REFERENCE TO RELATED APPLICATIONS

This application is a national phase entry of PCT Application No. PCT/JP2019/047438, filed on Dec. 4, 2019, which application is hereby incorporated herein by reference.

### TECHNICAL FIELD

The present invention relates to a heartbeat detection device, a heartbeat detection method, and a heartbeat detection program.

### BACKGROUND

In recent years, there has been a growing interest in self-health management by wearing wearable devices and using measurement data such as a footstep count, the amount of activity or a heart rate in various daily activity scenes such as running and walking.

For example, Patent Literature 1 discloses an application that uses a wearable sensor mounted on clothing to acquire an electrocardiographic waveform during wear, calculates a heart rate based on it, and sends obtained data to an external terminal such as a smartphone.

Conventionally-known wearable devices of this kind use no external power source, and thus, reduction in power consumption of the device is important in applications that require long-time continuous use such as measuring the heartbeat of a user.

In this regard, in order to achieve reduction in power consumption of the wearable device, there has conventionally been an attempt to reduce the computation load per unit time by increasing the interval of acquiring data such as the heart rate of the user.

For example, Patent Literature 2 discloses a technique for calculating the heart rate from the electrocardiographic potential by setting a threshold corresponding to the amplitude of an R wave for time-series data of the electrocardiographic potential, detecting the R wave when the data exceeds the threshold, and calculating the heart rate from its period (R-R interval).

Also, as a method for stably detecting the heart rate of a living body without influence by motion artifact noise, there has been proposed a technique of using, as an index value, a temporal difference value of the electrocardiographic potential or a temporal difference value obtained by considering clearances before and after peaks of the QRS waves, instead of using time-series data of the electrocardiographic potential (see Patent Literature 3).

### CITATION LIST

### Patent Literature

Patent Literature 1: International Publication No. WO 2016/024495

Patent Literature 2: Japanese Patent Laid-Open No. 2015-156936

Patent Literature 3: Japanese Patent No. 6404784

### SUMMARY

### Technical Problem

However, in conventional techniques, since a feature value of the electrocardiographic potential is detected by a threshold, shifts in detection times corresponding to the interval of data acquisition may occur, resulting in an error in the calculation of the heart rate.

Embodiments of the present invention have been made in order to solve the above-described problem, and it is an objective of embodiments of the present invention to more accurately measure the heart rate of a living body.

### Means for Solving the Problem

In order to solve the above-described problem, a heartbeat detection device according to embodiments of the present invention includes: a sensor data acquisition unit that acquires sensor data indicating an electrocardiographic waveform of a living body and outputs a sampling data sequence based on the sensor data; a first calculation unit that calculates a temporal difference value of sampling data from the output sampling data sequence for each sampling time; a determination unit that determines a heartbeat time on the basis of a time at which a change in the temporal difference value calculated by the first calculation unit exceeds a set threshold; a correction unit that corrects the heartbeat time by using a reference time that is a time at which the temporal difference value becomes zero on a straight line passing through two points of a temporal difference value of sampling data at the heartbeat time determined by the determination unit and a temporal difference value of immediately preceding sampling data; and a second calculation unit that calculates a heart rate of the living body from the heartbeat time corrected by the correction unit.

In order to solve the above-described problem, a heartbeat detection method according to embodiments of the present invention includes: a first step of acquiring sensor data indicating an electrocardiographic waveform of a living body and outputs a sampling data sequence based on the sensor data; a second step of calculating a temporal difference value of sampling data from the sampling data sequence output in the first step for each sampling time; a third step of determining a heartbeat time on the basis of a time at which a change in the temporal difference value calculated in the second step exceeds a set threshold; a fourth step of correcting the heartbeat time by using a reference time that is a time at which the temporal difference value becomes zero on a straight line passing through two points of a temporal difference value of sampling data at the heartbeat time determined in the third step and a temporal difference value of immediately preceding sampling data; and a fifth step of calculating a heart rate of the living body from the heartbeat time corrected in the fourth step.

In order to solve the above-described problem, a heartbeat detection program according to embodiments of the present invention is characterized by causing performance of: a first step of acquiring sensor data indicating an electrocardiographic waveform of a living body and outputs a sampling data sequence based on the sensor data; a second step of calculating a temporal difference value of sampling data from the sampling data sequence output in the first step for each sampling time; a third step of determining a heartbeat time on the basis of a time at which a change in the temporal difference value calculated in the second step exceeds a set threshold; a fourth step of correcting the heartbeat time by using a reference time that is a time at which the temporal difference value becomes zero on a straight line passing through two points of a temporal difference value of sampling data at the heartbeat time determined in the third step and a temporal difference value of immediately preceding sampling data; and a fifth step of calculating a heart rate of the living body from the heartbeat time corrected in the fourth step.

### Effects of Embodiments of the Invention

According to embodiments of the present invention, a heartbeat time is corrected by using a reference time that is a time at which a temporal difference value becomes zero on a straight line passing through two points of a temporal difference value of sampling data at a heartbeat time determined on the basis of a time at which change in the temporal difference value of the sampling data for each sampling time calculated from a sampling data sequence based on an electrocardiographic waveform exceeds a set threshold and a temporal difference value of immediately preceding sampling data. Thus, it is possible to more accurately measure the heart rate of a user.

### BRIEF DESCRIPTION OF THE DRAWINGS

FIG. 1 is a block diagram showing a functional configuration of a heartbeat detection device according to an embodiment of the present invention.

FIG. 2 is a diagram for illustrating an electrocardiographic waveform according to the present embodiment.

FIG. 3 is a diagram for illustrating an electrocardiographic waveform according to the present embodiment.

FIG. 4 is a block diagram showing a functional configuration of a heartbeat time calculation unit according to the present embodiment.

FIG. 5 is a block diagram showing another example of functional configuration of the heartbeat time calculation unit according to the present embodiment.

FIG. 6 is a block diagram showing an example of hardware configuration of the heartbeat detection device according to the present embodiment.

FIG. 7 is a flow chart for illustrating a heartbeat detection method according to the present embodiment.

FIG. 8 is a flow chart for illustrating a heartbeat time calculation process according to the present embodiment.

FIG. 9 is a diagram for illustrating effects of the heartbeat detection device according to the present embodiment.

### DETAILED DESCRIPTION OF ILLUSTRATIVE EMBODIMENTS

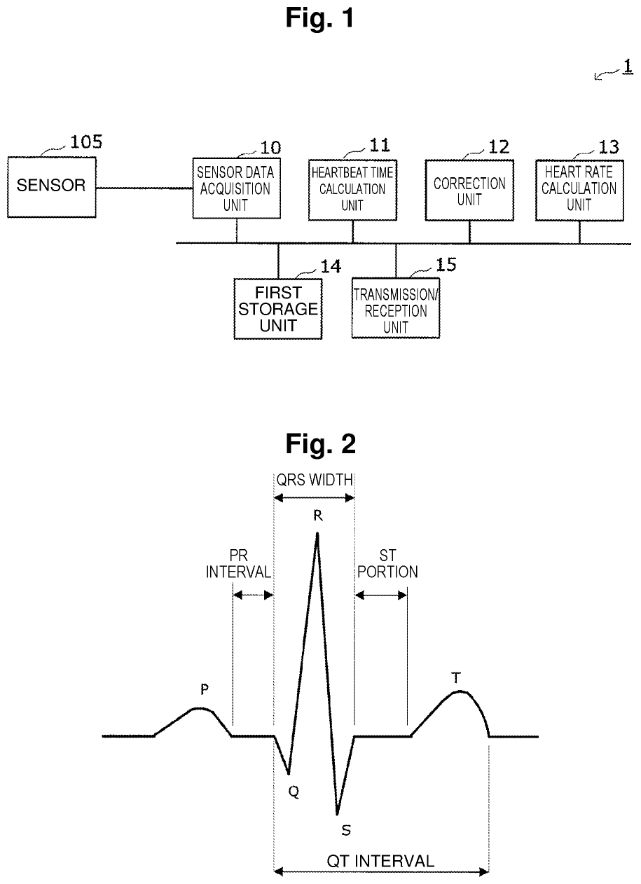

A preferred embodiment of the present invention will be described in detail below with reference to FIGS. 1 to 9.

### Outline of Embodiments of Invention

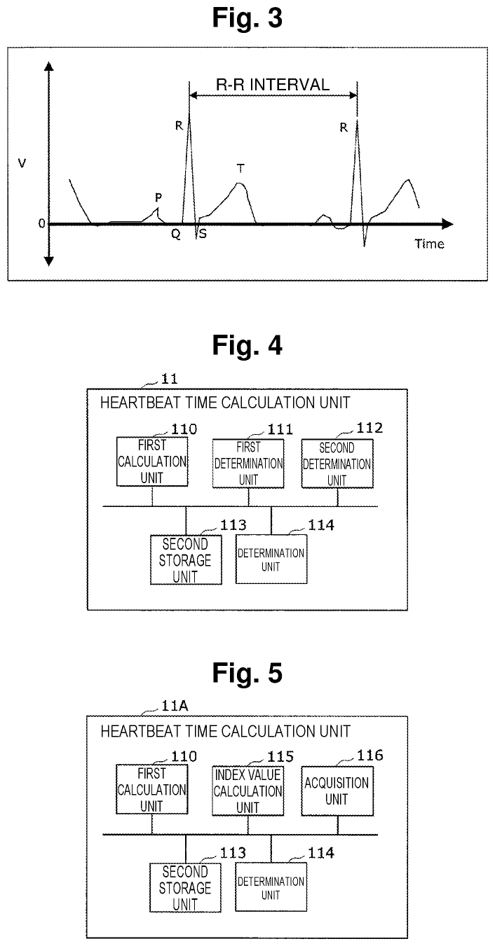

First, an outline of a heartbeat detection device 1 according to an embodiment of the present invention will be described with reference to FIGS. 2 and 3.

FIGS. 2 and 3 are diagrams showing general electrocardiographic waveforms (Electrocardiogram: ECG). An ECG waveform consists of a continuous heartbeat waveform, and one heartbeat waveform consists of components such as P, Q, R, S and T waves, which reflect activities of the atriums and ventricles, as shown in FIG. 2. Among them, an R wave occur in association with a contraction of the ventricles (depolarization of the ventricular muscle) and has a large amplitude, and therefore the detection of heartbeats is often performed with reference to R waves. In particular, it becomes easier to detect heartbeats by obtaining temporal differences in a sampling data sequence of the ECG waveform to emphasize steep changes from R waves to S waves in peaked shapes. The interval of each heartbeat is referred to as an R-R interval and is used as a primary index of heartbeat fluctuation.

The heartbeat detection device 1 according to the present embodiment uses a detection point on an RS interval from the starting portion of a QRS wave to the peak of an S wave included in the ECG waveform in order to detect a steep change from the R wave to the S wave. The present embodiment has a feature in using a temporal difference value of electrocardiographic potential corresponding to the detection point on the RS interval and its immediately preceding point to estimate times at which this temporal difference value becomes 0 in the RS interval and correcting the R-R interval by using the time interval of the estimated times.

Next, a configuration of the heartbeat detection device 1 according to the present embodiment will be described by using FIG. 1. The following describes a procedure in which the heartbeat detection device 1 detects one heartbeat and obtains its heartbeat time. By repeating such calculation of a heartbeat time for a period of measurement of the ECG waveform, time-series data of heartbeat times can be obtained, and the R-R interval can be calculated from this time-series data.

In the present embodiment, sampling data obtained by sampling the ECG waveform is defined as D(i). Here, i (i=1, 2, . . . ) indicates a number serially given to data obtained by one time of sampling. A larger number i of the data sequence indicates a later sampling time.

In the present embodiment, a minimum value of the temporal difference value in the range of a first time interval Tinr1, which will be described later, is defined as Min1, a minimum value of the temporal difference value in the range of a second time interval Tinr2 is defined as Min2, and a minimum value of the temporal difference value in the range of a third time interval Tinr3 is defined as Min3. The initial values of these minimum values Min1, Min2 and Min3 may be set to 0, for example.

### Functional Blocks of Heartbeat Detection Device

As shown in FIG. 1, the heartbeat detection device 1 includes a sensor data acquisition unit 10, a heartbeat time calculation unit 11, a correction unit 12, a heart rate calculation unit (second calculation unit) 13, a first storage unit 14, and a transmission/reception unit 15.

The sensor data acquisition unit 10 acquires sensor data including an electrocardiographic signal of a user and a measurement time, for example. The sensor data acquisition unit 10 can acquire an electrocardiographic signal of the user measured by a sensor 105 constituted by an electrocardiograph, for example. The sensor data acquisition unit 10 performs signal processing such as amplification, AD conversion, and noise removal on the acquired electrocardiographic signal. The sensor data acquisition unit 10 stores sampling data D(i) as time-series data of the ECG and a sampling time in the first storage unit 14. Note that, although the case where the sensor data acquisition unit 10 acquires an electrocardiographic signal of the user from the sensor 105 is described in the present embodiment, the sensor data acquisition unit 10 can also perform off-line acquisition of an electrocardiographic signal of the user measured in advance.

The heartbeat time calculation unit 11 reads the sampling data D(i) of the ECG waveform stored in the first storage unit 14 and calculates a heartbeat time. As shown in FIG. 4, the heartbeat time calculation unit 11 includes a first calculation unit 110, a first determination unit 111, a second determination unit 112, a second storage unit 113, and a determination unit 114 (see Patent Literature 3).

The first calculation unit 110 calculates a temporal difference value DY(i) of the sampling data D(i) of the ECG waveform for each sampling time (time indicated by i). More specifically, the first calculation unit 110 reads data D(i+1) of the next sampling to the sampling data D(i) of the ECG waveform and sampling data D(i−1) of the previous sampling from the first storage unit 14 and calculates the temporal difference value DY(i) of the sampling data D(i) based on the following equation (1).

DY(i)=D(i+1)−D(i−1) (1)

The first determination unit 111 determines whether the temporal difference value DY(i) calculated by the first calculation unit 110 is below a threshold TH. In the present embodiment, a peak of the temporal difference value DY(i) due to a steep change from an R wave to an S wave is detected, as shown in the ECG waveforms in FIGS. 2 and 3. The peak of the temporal difference value DY(i) appears as a negative value. Therefore, the first determination unit 111 performs threshold processing of the temporal difference value DY(i) by using a threshold TH that is a set negative value.

For example, the first determination unit 111 can use, as the threshold TH, a value obtained by multiplying by 0.5 an average value of peaks (Min2), e.g., five peaks (Min2), closest to the temporal difference value DY(i) detected as a heartbeat. The reason for multiplying the average value of the peaks (Min2) by 0.5 is to set the absolute value of the threshold TH to be smaller than that for the purpose of simply detecting peaks (see Non-Patent Literature 3). It is possible to capture a peak with a small absolute value by setting a smaller absolute value of the threshold TH.

The second determination unit 112 determines whether a first elapsed time T1 from the immediately preceding heartbeat time to the latest sampling time (the time indicated by i) at which the temporal difference value DY(i) is obtained is in the range of a first time interval Tinr1. The second determination unit 112 also determines whether a second elapsed time T2 from the time at which the temporal difference value DY(i) falls below the threshold TH to the latest sampling time at which the temporal difference value DY(i) is obtained is in the range of a second time interval Tinr2. Further, the second determination unit 112 determines whether a third elapsed time T3 from the time at which the second elapsed time T2 exceeds the range of the second time interval Tinr2 to the latest sampling time at which the temporal difference value DY(i) is obtained is in the range of a third time interval Tinr3.

The first time interval Tinr1 is a parameter for defining a time region before the next assumed heartbeat time. The second time interval Tinr2 is a parameter for defining a time region assumed to include a peak of the temporal difference value DY(i). The third time interval Tinr3 is a parameter for defining a certain time region after the time region assumed to include the peak of the temporal difference value DY(i).

For example, the second determination unit 112 uses, as the range of first time interval Tinr1, a range between a time length shorter by 150 [ms] than the R-R interval obtained from the immediately preceding heartbeat time and a time length obtained by adding 100 [ms] to this time length. The R-R interval here is a time length obtained by subtracting the second preceding heartbeat time from the immediately preceding heartbeat time.

Alternatively, the second determination unit 112 can use, as the range of the first time interval Tinr1, a time region from the immediately preceding heartbeat time to immediately before the temporal difference value DY(i) next exceeds the threshold TH.

The second time interval Tinr2 is preferably a time width sufficient to cover a peak of the temporal difference value DY(i) and can be preset to 50 [ms], for example.

The third time interval Tinr3 can be preset to 100 [ms], for example.

The second storage unit 113 retains a minimum value Mini of the temporal difference value DY(i) when the first elapsed time T1 is in the range of the first time interval Tinr1. The second storage unit 113 also retains a minimum value Min2 of the temporal difference value DY(i) when the second elapsed time T2 is in the range of the second time interval Tinr2. The second storage unit 113 also retains a minimum value Min3 of the temporal difference value DY(i) when the third elapsed time T3 is in the range of the third time interval Tinr3.

The determination unit 114 regards, as a heartbeat time, a time at which the temporal difference value DY(i) falls below the threshold TH or a time at which the minimum value Min2 is obtained when the relationship between the minimum values Min1, Min2 and Min3 of the temporal difference value DY(i) retained by the second storage unit 113 satisfies a preset condition for fixing the heartbeat time. Which time the determination unit 114 adopts as the heartbeat time can be preset by a program.

For example, the condition that both of a ratio Min2/Min1 of the minimum value Min2 to the minimum value Min1 and a ratio Min2/Min3 of the minimum value Min2 to the minimum value Min3 exceed a predetermined value can be used as the preset condition for fixing the heartbeat time. By presetting such a condition, it is possible to determine whether or not the peak of the temporal difference value is unimodal.

The correction unit 12 identifies a time at which the temporal difference value of the ECG waveform data becomes o from ECG waveform data based on the heartbeat time calculated by the heartbeat time calculation unit 11. In addition, the correction unit 12 uses the time at which the temporal difference value becomes o in the ECG waveform data as a reference time for calculating the R-R interval. More specifically, the correction unit 12 identifies a time at which the temporal difference value becomes 0, that is, the time of an R wave included in the ECG waveform on a straight line passing through two points of a temporal difference value DY(n) of ECG waveform data D(n) at the heartbeat time calculated by the heartbeat time calculation unit 11 and the immediately preceding temporal difference value DY(n−1), and regards it as a reference time for calculating the R-R interval.

For example, a shift Δn (the unit is the sampling data number) with reference to a heartbeat time at which the electrocardiographic potential becomes 0 is calculated by the following equation (2).

$$\begin{matrix}Math 1 & \\ Δ n = - \frac{DY ( n ) - DY ( n - 1 )}{DY ( n )} & ( 2 )\end{matrix}$$

The correction unit 12 can calculate the reference time by using the shift Δn calculated from the above equation (2).

Here, it is necessary that the ECG waveform data D(n) at the heartbeat time calculated by the heartbeat time calculation unit 11 and the immediately preceding ECG waveform data D(n−1) are measurement points on an interval from an inflection point immediately before the R wave to an inflection point immediately after the R wave. That is, there is an upper limit of the sampling interval. For example, in consideration of the fact that the QRS interval is generally said to be about 100 [ms], the above-mentioned interval is estimated to be 25 [ms], and thus the sampling interval needs to be 12.5 [ms] or less.

On the other hand, if the sampling interval is too short, the ECG waveform data D(n) at the heartbeat time calculated by the heartbeat time calculation unit 11 and the immediately preceding ECG waveform data D(n−1) would be present around the inflection point immediately after the R wave, that is, the temporal difference value DY(n) of the ECG waveform and the immediately preceding temporal difference value DY(n−1) would be present in a region where the gradient of the tangent to the time-series data of the temporal difference value steeply changes. Thus, a large error occurs when the heartbeat time is corrected by using the two points of the temporal difference value DY(n) of the ECG waveform and the immediately preceding temporal difference value DY(n−1). That is, there is also a lower limit of the sampling interval, which is specifically about 4 [ms].

The heart rate calculation unit 13 calculates a heart rate X [bpm] from the latest heartbeat time corrected by the correction unit 12. Specifically, the heart rate calculation unit 13 calculates an instantaneous heart rate X by the following equation (3), where the R-R interval, which is a time length obtained by subtracting from the latest heartbeat time calculated and fixed by the correction unit 12 its preceding heartbeat time, is defined as RRI [ms].

$$\begin{matrix}Math 2 & \\ X = \frac{60000}{RRI} & ( 3 )\end{matrix}$$

The heart rate calculation unit 13 can also calculate an average heart rate X by using the following equation (4) described in reference literature 3 (Japanese Patent Laid-Open No. 2018-011819), instead of the instantaneous heart rate.

Math 3

X(i)=q×HR(i)+(1−q)×X(i−1) (4)

In the above equation (4), HR(i) represents the i-th instantaneous heart rate before averaging, X(i−1) represents a value obtained by averaging the instantaneous heart rates up to the (i−1)-th, 1 represents a predetermined averaging coefficient, and X(i) represents an average heart rate obtained by averaging the instantaneous heart rates up to the i-th.

The transmission/reception unit 15 sends the heart rate calculated by the heart rate calculation unit 13 to an external terminal device such as an external smartphone (not shown) in a wired or wireless manner.

### Another Example of Heartbeat Time Calculation Unit

The heartbeat detection device 1 according to the present embodiment can also calculate the heartbeat time by a heartbeat time calculation unit 11A having a configuration shown in FIG. 5, for example, without limitation to the functional configuration of the heartbeat time calculation unit 11 described above (see reference literature 1: Japanese Patent No. 6360017 and reference literature 2: Japanese Patent No. 6527286).

The heartbeat time calculation unit 11A includes a first calculation unit 110, an index value calculation unit 115, an acquisition unit 116, a second storage unit 113, and a determination unit 114.

The first calculation unit no calculates a temporal difference value DY(i) of the sampling data D(i) of the electrocardiographic waveform for each sampling time.

For each sampling point i, the acquisition unit 116 acquires the minimum value of the temporal difference value in a predetermined time region before and after the sampling point i.

For each sampling point i, the index value calculation unit 115 obtains, as an index value, a value obtained by subtracting the minimum value of the temporal difference value in the predetermined time region before and after the sampling point i from the temporal difference value DY(i) of the sampling point i.

The determination unit 114 identifies, as a downward peak, an index value of the index values of respective sampling points i that is below a predetermined threshold and is at a point at which the tendency of change of the index value turns from decrease to increase, and regards the time of the identified downward peak as a heartbeat time.

For example, the predetermined time region before and after the sampling point i is a region of −112.5 ms to −12.5 ms and a region of +12.5 ms to +112.5 ms with respect to the time at the sampling point i.

The second storage unit 113 temporarily stores the minimum value of the temporal difference value acquired by the acquisition unit 116, the index value obtained by the index value calculation unit 115, and the heartbeat time determined by the determination unit 114.

### Hardware Configuration of Heartbeat Detection Device

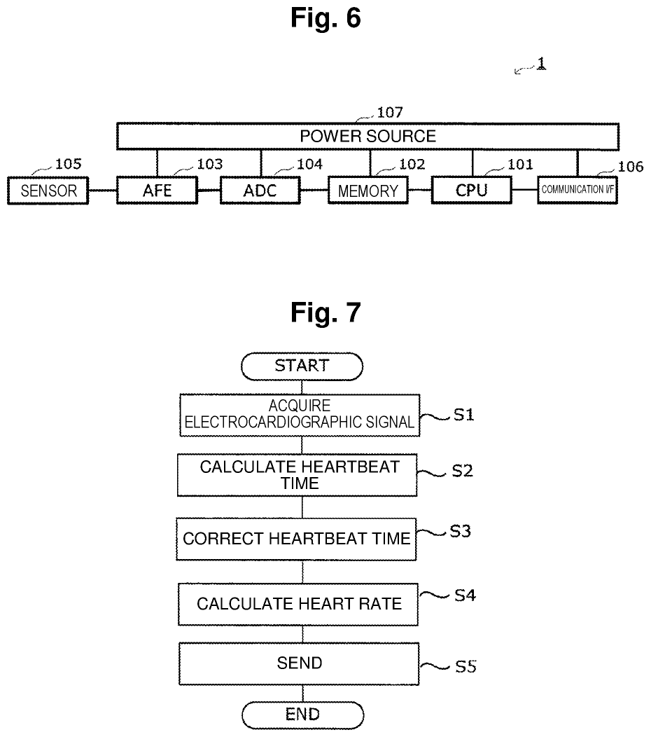

Next, an example of hardware configuration for realizing the heartbeat detection device 1 having the above-described functions will be described with reference to FIG. 6.

As shown in FIG. 6, the heartbeat detection device 1 can be realized by a computer including a CPU 101, a memory 102, an AFE 103, an ADC 104, and a communication I/F 106 connected via a bus and programs for controlling these hardware resources, for example. For example, a sensor 105 provided externally is connected to the heartbeat detection device 1 via the bus. The heartbeat detection device 1 also includes a power source 107 and provides power supply to the entire device except for the sensor 105 as shown in FIG. 5.

Programs for performing various control and computational operations by the CPU 101 are stored in the memory 102 in advance. The functions of the heartbeat detection device 1 including the sensor data acquisition unit 10, the heartbeat time calculation unit 11, the correction unit 12 and the heart rate calculation unit 13 shown in FIG. 1 are realized by the CPU 101 and the memory 102.

The sensor 105 is realized by an electrocardiograph or the like and measures weak electrocardiographic signals through the skin of the user.

The AFE (Analog Front End) 103 is a circuit for amplifying the analog electrocardiographic signals measured by the sensor 105.

The ADC (Analog-to-Digital Converter) 104 is a circuit for converting the analog signals amplified by the AFE 103 into digital signals with a predetermined sampling frequency. The AFE 103 and the ADC 104 realize the sensor data acquisition unit 10 illustrated in FIG. 1.

The memory 102 is realized by a non-volatile memory such as a flash memory, a volatile memory such as a DRAM, or the like. The memory 102 temporarily stores time-series data of signals output from the ADC 104. The memory 102 realizes the first storage unit 14 illustrated in FIG. 1 and the second storage unit illustrated in FIG. 5.

In addition, the memory 102 has a program storage region for storing programs for performing the heartbeat detection process by the heartbeat detection device 1. Further, for example, it may also have a backup region for backing up the above-described data and programs and the like.

The communication I/F 106 is a interface circuit for performing communication with various external electronic devices via a communication network NW.

As the communication I/F 106, a communication interface or an antenna compatible with wired or wireless data communication standards such as LTE, 3G, Bluetooth (R), Bluetooth Low Energy, and Ethernet (R), for example, is used. The transmission/reception unit 15 illustrated in FIG. 1 is realized by the communication I/F 106.

Note that the heartbeat detection device 1 acquire time information from a clock integrated in the CPU 101 or a time server (not shown) and uses it as a sampling time.

### Heartbeat Detection Method

Next, operations of the heartbeat detection device 1 having the above-described configuration will be described by using the flow chart in FIG. 7. First, when the sensor 105 consisting of an electrocardiograph is worn on the user and measurement of electrocardiographic signals is started, the following process is performed.

First, the sensor data acquisition unit 10 acquires an electrocardiographic signal of the user, for example, from the sensor 105 (step S1). The sensor data acquisition unit 10 amplifies the electrocardiographic signal, samples it with a predetermined sampling frequency, and outputs a digital ECG waveform. The sampling data of the ECG waveform is stored in the first storage unit 14.

Next, the heartbeat time calculation unit 11 reads the sampling data D(i) of the ECG waveform from the first storage unit 14 and calculates a heartbeat time (step S2).

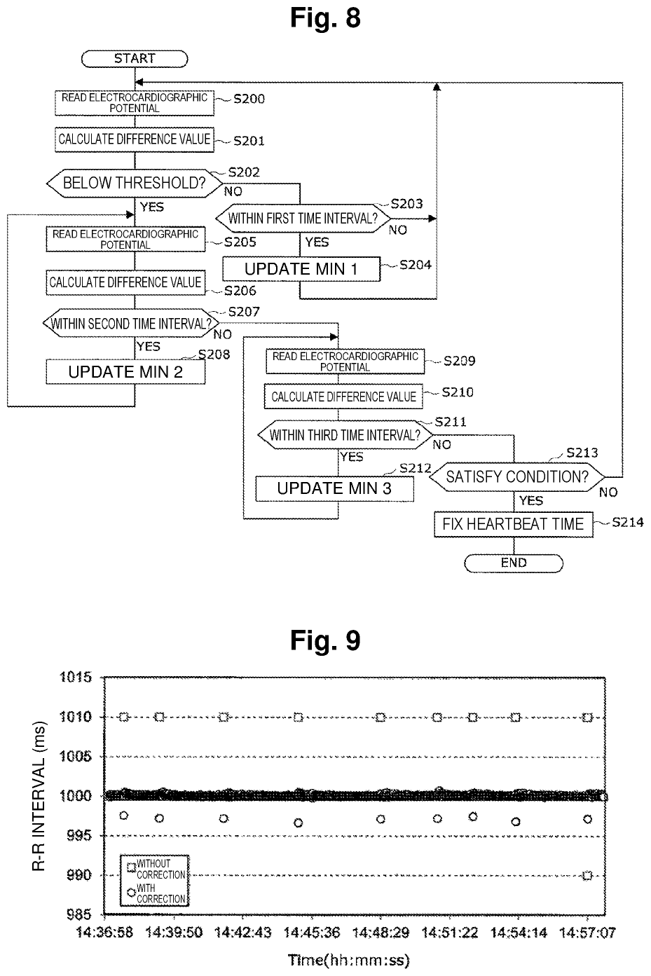

Here, a heartbeat time calculation process performed by the heartbeat time calculation unit 11 in step S2 will be described with reference to FIG. 8.

First, the first calculation unit 110 reads data D(i+1) of the next sampling to the sampling data D(i) and data D(i−1) of the previous sampling in order to calculate the temporal difference value DY(i) of the sampling data D(i) from the first storage unit 14 (step S200).

Next, the first calculation unit no calculates the temporal difference value DY(i) of the sampling data D(i) from equation (1) (step S201). Next, the first determination unit 111 determines whether the temporal difference value DY(i) is below the threshold TH (step S202).

Next, when it is determined that the temporal difference value DY(i) is not below the threshold TH (step S202: NO), the second determination unit 112 determines whether the first elapsed time T1, which is the elapsed time from the immediately preceding heartbeat time to the sampling time (the time indicated by i) of the sampling data D(i) to be processed, is in the range of the first time interval Tinr1 (step S203).

When it is determined that the first elapsed time T1 is in the range of the first time interval Tinr1, that is, when the sampling time of the sampling data D(i) to be processed is in a time region before the next assumed heartbeat time (step S203: YES), the second storage unit 113 updates the minimum value Mini of the temporal difference value in the range of the first time interval Tinr1 (step S204). That is, the second storage unit 113 compares the temporal difference value DY(i) of the sampling data D(i) to be processed and the current minimum value Min1, and when the temporal difference value DY(i) to be processed is smaller than the current minimum value Min1, sets the temporal difference value DY(i) as a new minimum value Min1. The process then returns to step S200. Note that, when it is determined in step S203 that the first elapsed time T1 is out of the range of the first time interval Tinr1, the process returns to step S200 without updating the minimum value Min1.

On the other hand, when it is determined that the temporal difference value DY(i) exceeds the threshold TH (step S202: YES), the first calculation unit 110 reads data D(j+1) of the next sampling to sampling data D(j) and data D(j−1) of the previous sampling from the first storage unit 14 in order to calculate a temporal difference value DY(j) of the next sampling data D(j) (j is an integer of j≥(i+1) and its initial value is j=i+1) (step S205). Then, the first calculation unit 110 calculates the temporal difference value DY(j) of the sampling data D(j) in the same way as the above equation (1) (step S206).

A time determination unit 5 determines whether the elapsed time from the time at which the temporal difference value Y(i) exceeds the threshold TH to the sampling time (the time indicated by i) of the sampling data X(j) to be processed (the second elapsed time T2) is in the range of the second time interval Tinr2 (step S207).

When it is determined that the second elapsed time T2 is in the range of the second time interval Tinr2, that is, when the sampling time of the sampling data D(j) to be processed is in a time region assumed to include a peak of the temporal difference value (step S207: YES), the second storage unit 113 updates the minimum value Min2 of the temporal difference value in the range of the second time interval Tinr2 (step S208). That is, the second storage unit 113 compares the temporal difference value DY(j) of the sampling data D(j) to be processed and the current minimum value Min2, and when the temporal difference value DY(j) to be processed is smaller than the current minimum value Min2, sets the temporal difference value DY(j) as a new minimum value Min2. The process then returns to step S205.

Thus, the processes of steps S205 to S208 are repeatedly performed while shifting the processing target toward sampling data D(j) of a newer sampling time such that j=i+1, i+2, i+3, i+4, . . . until the second elapsed time T2 is out of the range of the second time interval Tinr2.

Next, when it is determined in step S207 that the second elapsed time T2 exceeds the range of the second time interval Tinr2, that is, when the sampling time exceeds the time region assumed to include the peak of the temporal difference value, the first calculation unit no reads data D(k+1) of the next sampling to sampling data D(k) and data D(k−1) of the previous sampling from the first storage unit 14 in order to calculate a temporal difference value DY(k) of the next sampling data D(k) (k is an integer of k≥(j+1) and its initial value is k=j+1) (step S209). Then, the first calculation unit no calculates the temporal difference value DY(k) of the sampling data D(k) in the same way as the above equation (1) (step S210).

The second determination unit 112 determines whether the elapsed time from the time at which it is determined in step S207 that the second elapsed time T2 exceeds the range of the second time interval Tinr2 to the sampling time (the time indicated by k) of the sampling data D(k) to be processed (the third elapsed time T3) is in the range of the third time interval Tinr3 (step S211).

When it is determined that the third elapsed time T3 is in the range of the third time interval Tinr3, that is, when the sampling time of the sampling data D(k) to be processed is in a certain time region after the peak of the temporal difference value (step S211: YES), the second storage unit 113 updates the minimum value Min3 of the temporal difference value in the range of the third time interval Tinr3 (step S212). That is, the second storage unit 113 compares the temporal difference value DY(k) of the sampling data D(k) to be processed and the current minimum value Min3, and when the temporal difference value DY(k) to be processed is smaller than the current minimum value Min3, sets the temporal difference value DY(k) as a new minimum value Min3. The process then returns to step S209.

Thus, the processes of steps S209 to S212 are repeatedly performed while shifting the processing target toward sampling data D(k) of a newer sampling time such that k=j+1, j+2, j+3, j+4, . . . until the third elapsed time T3 is out of the range of the third time interval Tinr3.

Next, when it is determined in step S211 that the third elapsed time T3 is out of the range of the third time interval Tinr3, that is, when the sampling time of the sampling data D(k) to be processed exceeds the certain time region after the peak of the temporal difference value, the determination unit 114 determines whether the relationship between the three minimum values Min1, Min2 and Min3 satisfies a predetermined condition for fixing the heartbeat time (step S213).

Here, the minimum value Min2 corresponds to the magnitude of the detected peak of the temporal difference value of the sampling data, the minimum value Min1 corresponds to a floor level in a certain region before the peak, and the minimum value Min3 corresponds to a floor level in a certain region after the peak. If the peak of the temporal difference value is a unimodal negative peak, the minimum value Min2 must have a larger absolute value than the minimum values Min1 and Min3.

The determination unit 114 can determine that the predetermined condition is satisfied when the ratios Min2/Min1 and Min2/Min3 both exceed a predetermined value and determine that the predetermined condition is not satisfied when at least one of the ratios Min2/Min1 and Min2/Min3 is less than or equal to the predetermined value.

When it is determined that the relationship between the three minimum values Min1, Min2 and Min3 satisfies the condition for fixing the heartbeat time (step S213: YES), the determination unit 114 adopts, as a heartbeat time, a time at which the temporal difference value DY(i) exceeds the threshold TH or the latest time at which the minimum value Min2 is updated (the time of the peak of the temporal difference value) (step S214).

After step S214 ends, the process returns to step S200 by setting i=k+1. Then, detection of the next heartbeat is started. In addition, although the process also returns to step S200 by setting i=k+1 when it is determined in step S213 that the relationship between the three minimum values Min1, Min2 and Min3 does not satisfies the condition for fixing the heartbeat time, in this case, the temporal difference value exceeding the threshold TH detected in step S202 is not due to a heartbeat but a noise, and the heartbeat is not detected yet.

By repeating the processes of steps S200 to S214 in this manner, time-series data of heartbeat times can be obtained.

Next, returning to FIG. 7, the correction unit 12 corrects the heartbeat time obtained by the heartbeat time calculation unit 11 (step S3). More specifically, the correction unit 12 identifies a time at which the temporal difference value of the ECG waveform becomes o from ECG waveform data based on the heartbeat time calculated by the heartbeat time calculation unit 11. In addition, the correction unit 12 uses the time at which the identified temporal difference value becomes 0 as a reference time for calculating the R-R interval. More specifically, the correction unit 12 identifies a time at which the temporal difference value becomes 0 on a straight line passing through two points of an immediately preceding temporal difference value DY(n) in the ECG waveform data at the heartbeat time calculated by the heartbeat time calculation unit 11 and the immediately following temporal difference value DY(n−1), and regards it as a reference time for calculating the R-R interval. The time at which the temporal difference value becomes 0 in the ECG waveform identified by the correction unit 12 is the time of an R wave in the ECG waveform.

Next, the heart rate calculation unit 13 calculates the heart rate X [bpm] from the latest heartbeat time corrected by using the reference time, at which the temporal difference value becomes 0, identified by the correction unit 12 (step S4). More specifically, the heart rate calculation unit 13 calculates an instantaneous heart rate X by equation (3) mentioned above, where the R-R interval, which is a time obtained by subtracting from the latest heartbeat time calculated and fixed by the correction unit 12 its preceding heartbeat time, is defined as RRI [ms]. Alternatively, the heart rate calculation unit 13 can also calculate an average heart rate X by using equation (4) mentioned above, instead of the instantaneous heart rate.

Subsequently, the transmission/reception unit 15 sends the instantaneous heart rate X or average heart rate X calculated by the heart rate calculation unit 13 to an external terminal (not shown) such as a smartphone via a communication network in a wired or wireless manner (step S5).

### Effects of Heartbeat Detection Device

Next, effects of the heartbeat detection device 1 according to the present embodiment will be described with reference to FIG. 9. The example of FIG. 9 shows detection results of the R-R interval (RRI) in the presence and absence of the correction unit 12 when an electrocardiographic waveform with a constant heart rate of 60 [bpm] is input. In FIG. 9, the horizontal axis indicates time, and the vertical axis indicates the R-R interval. “Quadrangular” markers indicate measurement results by a heartbeat detection device not including the correction unit 12 according to a conventional example. On the other hand, “circular” markers indicate measurement results by the heartbeat detection device 1 including the correction unit 12 according to the present embodiment.

In the example of FIG. 9, heartbeat times calculated by the heartbeat time calculation unit 11A described in reference literature 1 and reference literature 2 as the heartbeat time calculation unit 11 are used. In addition, the sampling interval is 10 [ms]. It can be seen from the calculation results in FIG. 9 that detect errors are reduced due to the introduction of the correction unit 12. In addition, it is demonstrated that the heartbeat detection device 1 according to the present embodiment can detect the heartbeat and the RRI with an accuracy exceeding the sampling interval from the ECG waveform.

As described above, with the heartbeat detection device 1 according to the present embodiment, since a time at which the temporal difference value becomes 0, that is, the time of an R wave in the electrocardiographic waveform is identified on a straight line passing through two points of the temporal difference value DY(n) of the electrocardiographic waveform at the heartbeat time calculated by the heartbeat time calculation unit 11 and the immediately preceding temporal difference value DY(n−1) and the R-R interval is corrected by using the identified time, it is possible to more accurately measure the heart rate of a living body.

In addition, according to the present embodiment, it is possible to accurately detect the heart rate of the user even when the interval of data acquisition for acquiring the heart rate of the user with an electrocardiograph is set to be larger in order to reduce power consumption.

Note that, although a case of detecting the heart rate is illustrated in the described embodiment, there is no limitation to the heart rate, and application is also possible for calculating the heart rate on the basis of biological information with periodicity such as brain waves and for correcting other feature values of the R-R interval included in the ECG waveform, for example, sample data such as P, Q, S and T waves.

In addition, in the described embodiment, a case of using the techniques described in Patent Literature 3 and reference literatures 1 and 2 has been described as a specific example of the heartbeat time calculation unit 11. However, the heartbeat time calculation unit 11 is not limited to the heartbeat time calculation method described in these literatures, and the heartbeat detection device 1 according to the present embodiment can be applied.

In addition, the heartbeat time calculation method described in Patent Literature 3 and reference literatures 1 and 2 can reduce erroneous heartbeat detection caused by fluctuation of the base line or a steep noise such as due to body motions and sweating as compared to the conventional heartbeat detection techniques. Thus, by combining the heartbeat time calculation method described in Patent Literature 3 and reference literatures 1 and 2 and the correction unit 12 according to embodiments of the present invention, it is possible to calculate the heart rate with a higher accuracy.

Although the embodiment of the heartbeat detection device, the heartbeat detection method, and the heartbeat detection program of the present invention has been described above, the present invention is not limited to the described embodiment, and various modifications are possible that can be conceived by those skilled in the art within the scope of the invention defined in the claims.

### REFERENCE SIGNS LIST

1 Heartbeat detection device
10 Sensor data acquisition unit
11, 11A Heartbeat time calculation unit
12 Correction unit
13 Heart rate calculation unit
14 First storage unit
15 Transmission/reception unit
110 First calculation unit
111 First determination unit
112 Second determination unit
113 Second storage unit
114 Determination unit
115 Index value calculation unit
116 Acquisition unit
101 CPU
102 Memory
103 AFE
104 ADC
105 Sensor
106 Communication I/F
107 Power source.

1-7. (canceled)

8. A heartbeat detection device comprising:
a sensor data acquisition circuit configured to acquire sensor data indicating an electrocardiographic waveform of a living body and output a sampling data sequence based on the sensor data;
a first calculation circuit configured to calculate a temporal difference value of sampling data from the sampling data sequence for a plurality of sampling times;
a determination circuit configured to determine a heartbeat time on the basis of a time at which a change in the temporal difference value calculated by the first calculation circuit exceeds a set threshold;
a correction circuit configured to correct the heartbeat time in accordance with a reference time, the reference time being is a time at which the temporal difference value becomes zero on a straight line passing through two points of a first temporal difference value of sampling data at the heartbeat time and a second temporal difference value of an immediately preceding sampling data; and
a second calculation circuit configured to calculate a heart rate of the living body from the heartbeat time corrected by the correction circuit.

9. The heartbeat detection device according to claim 8, wherein
the two points of the sampling data at the heartbeat time and the immediately preceding sampling data are points on an interval from a first inflection point immediately before an R wave included in the electrocardiographic waveform to a second inflection point immediately after the R wave.

10. The heartbeat detection device according to claim 8, wherein the second calculation circuit is further configured to:
calculate an R-R interval that is a time interval of an R wave included in the electrocardiographic waveform of the living body and a preceding R wave in accordance with the reference time obtained by the correction circuit; and
calculate the heart rate of the living body from the R-R interval.

11. A heartbeat detection method comprising:
a first step of acquiring, by a sensor data acquisition circuit, sensor data indicating an electrocardiographic waveform of a living body and outputting a sampling data sequence based on the sensor data;
a second step of calculating, by a first calculation circuit, a temporal difference value of sampling data from the sampling data sequence output in the first step for a plurality of sample times;
a third step of determining, by a determination circuit, a heartbeat time on the basis of a time at which a change in the temporal difference value calculated in the second step exceeds a set threshold;
a fourth step of correcting, by a correction circuit, the heartbeat time by using a reference time, the reference time being a time at which the temporal difference value becomes zero on a straight line passing through two points of a first temporal difference value of sampling data at the heartbeat time determined in the third step and a second temporal difference value of an immediately preceding sampling data; and
a fifth step of calculating, a second calculation circuit, a heart rate of the living body from the heartbeat time corrected in the fourth step.

12. The heartbeat detection method according to claim 11, wherein the two points of the sampling data at the heartbeat time determined in the third step and the immediately preceding sampling data are points on an interval from a first inflection point immediately before an R wave included in the electrocardiographic waveform to a second inflection point immediately after the R wave.

13. The heartbeat detection method according to claim ii, wherein the fifth step comprises:
calculating an R-R interval, the R-R interval being a time interval of an R wave included in the electrocardiographic waveform of the living body and a preceding R wave by using the reference time obtained in the fourth step; and
calculating the heart rate of the living body from the R-R interval.

14. A heartbeat detection program that causes a computer to perform:
a first step of acquiring sensor data indicating an electrocardiographic waveform of a living body and outputting a sampling data sequence based on the sensor data;
a second step of calculating a temporal difference value of sampling data from the sampling data sequence output in the first step for a plurality of sample times;
a third step of determining a heartbeat time on the basis of a time at which a change in the temporal difference value calculated in the second step exceeds a set threshold;
a fourth step of correcting the heartbeat time by using a reference time, the reference time being a time at which the temporal difference value becomes zero on a straight line passing through two points of a first temporal difference value of sampling data at the heartbeat time determined in the third step and a second temporal difference value of an immediately preceding sampling data; and
a fifth step of calculating a heart rate of the living body from the heartbeat time corrected in the fourth step.
